(19) United States
(12) Patent Application Publication
(10) Pub. No.: US 2026/0183464 A1
(43) Pub. Date: Jul. 2, 2026

(54) URINE COLLECTION CONTAINER ASSEMBLIES AND RELATED SYSTEMS AND METHODS

(71) Applicant: PUREWICK CORPORATION, Covington, GA (US)

(72) Inventors: Michelle Tourchak, Alpharetta, GA (US); Vinayaka Kulkarni, Bangalore (IN); Claire Gloeckner, Lilburn, GA (US); Samuel Hernandez, Covington, GA (US); Jonathan Robichaud, Decatur, GA (US); Rebecca Weiss, Atlanta, GA (US); Seth Schneider, Social Circle, GA (US)

(21) Appl. No.: 19/131,440

(22) PCT Filed: Nov. 23, 2022
(86) PCT No.: PCT/US2022/050909

§ 371 Date: May 20, 2025

(51) Int. Cl. A61M 1/00; A61F 5/451

(52) U.S. Cl. CPC A61M 1/60; A61F 5/451; A61M 2207/00

(57) ABSTRACT

Examples relate to urine collection container assemblies. A urine collection container assembly a canister having an interior region, a lid, and a gasket. The lid is secured or securable to the canister and includes multiple ports configured to detachably secure to one or more conduits to provide fluid communication with the interior region of the canister. The gasket is overmolded to the lid and positioned to interface a portion of the canister to form a seal when the lid is secured to the canister.

### BACKGROUND

An individual may have limited or impaired mobility such that typical urination processes are challenging or impossible. For example, the individual may have surgery or a disability that impairs mobility. In another example, the individual may have restricted travel conditions such as those experienced by pilots, drivers, and workers in hazardous areas. Additionally, fluid collection from the individual may be needed for monitoring purposes or clinical testing.

Bed pans and urinary catheters, such as a Foley catheter, may be used to address some of these circumstances. However, bed pans and urinary catheters have several problems associated therewith. For example, bed pans may be prone to discomfort, spills, and other hygiene issues. Urinary catheters be may be uncomfortable, painful, and may cause urinary tract infections. Conventional urine collection devices also may be limited to use when a patient is confined to a bed in a supine position.

Thus, users and manufacturers of fluid collection devices continue to seek new and improved devices, systems, and methods to collect urine.

### SUMMARY

Embodiments disclosed herein are related to urine collection container assemblies. In an embodiment, a urine collection container assembly includes a canister having an interior region, a lid, and a gasket. The lid is secured or securable to the canister and includes a vacuum port configured to detachably secure to a first conduit to provide fluid communication between a pump and the interior region of the canister, and a urine port configured to detachably secure to a second conduit to provide fluid communication between a urine collection device and the interior region of the canister. The gasket is overmolded to the lid and positioned to interface a portion of the canister to form a seal when the lid is secured to the canister.

In an embodiment, a method of forming a lid for a urine collection container assembly is described. The method includes injection molding with a first material a lid having a rim, a vacuum port configured to detachably secure to a first conduit to provide fluid communication between a pump and the interior region of the canister, and a urine port configured to detachably secure to a second conduit to provide fluid communication between a urine collection device and the interior region of the canister. The method also includes overmolding with a second material a gasket to at least a portion of the rim of the lid, the second material being different than the first material.

In an embodiment, a urine collection container assembly is described. The urine collection container assembly includes a canister and a lid. The canister has an outer surface, an interior region, an upper region having an upper edge, and a locking rib on the outer surface of the canister at least proximate to the upper region of the canister. The lid is secured or securable to the canister and includes a vacuum port configured to detachably secure to a first conduit to provide fluid communication between a pump and the interior region of the canister, a urine port configured to detachably secure to a second conduit to provide fluid communication between a urine collection device and the interior region of the canister, and at least one clamp having an inward oriented projection configured to selectively engage with at least a portion of the locking rib to secure the lid to the canister.

In an embodiment, a urine collection system is described. The urine collection system includes any of the urine collection container assemblies described herein, a urine collection device, a pump, a first conduit secured to a vacuum port and in fluid communication with the pump and the interior region of the canister, and a second conduit secured to the urine port and in fluid communication with the urine collection device and the interior region of the canister.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a block diagram of a portable urine collection system, according to an embodiment.

FIG. 1B is a side view of a urine collection system in a urine collection configuration, according to an embodiment.

FIGS. 1C-1D are isometric side views of a urine collection container assembly, according to an embodiment.

FIGS. 1E-1F are isometric side views of a urine collection container assembly, according to an embodiment.

FIGS. 1G-1H are isometric side views of a urine collection container assembly, according to an embodiment.

FIGS. 1I-1J are isometric side views of a urine collection container assembly, according to an embodiment.

FIGS. 1K-1L are isometric side views of a urine collection container assembly, according to an embodiment.

FIGS. 1M-1O are isometric side views of a urine collection container assembly, according to an embodiment.

FIG. 1P is a partial cross-sectional view of an upper edge of the canister and the lid of the urine collection container assembly of FIGS. 1C-1D.

FIGS. 2A-2B are isometric side views of a urine collection container assembly, according to an embodiment.

FIGS. 2C-2D are isometric side views of a urine collection container assembly, according to an embodiment.

FIGS. 2E-2F are isometric side views of a urine collection container assembly, according to an embodiment.

FIGS. 2G-2H are isometric side views of a urine collection container assembly, according to an embodiment.

FIGS. 2I-2J are isometric side views of a urine collection container assembly, according to an embodiment.

FIGS. 3A-3B are isometric side views of a urine collection container assembly, according to an embodiment.

FIGS. 3C-3D are isometric side views of a urine collection container assembly, according to an embodiment.

FIGS. 3E-3F are isometric side views of a urine collection container assembly, according to an embodiment.

FIGS. 3G-3H are isometric side views of a urine collection container assembly, according to an embodiment.

FIG. 4A is an isometric side view of a urine collection container assembly, according to an embodiment.

FIG. 4B is a cross-sectional view of a clamping region of the urine collection container assembly of FIG. 4A.

FIG. 4C is a cross-sectional view of a spout region of the urine collection container assembly of FIG. 4A.

FIGS. 4D-4G are views of a clamping region of a urine collection container assembly, according to an embodiment.

FIG. 5 is an isometric side view of a urine collection container assembly, according to an embodiment.

FIG. 6A is an isometric side view of a urine collection container assembly, according to an embodiment.

FIG. 6B is an exploded view of the urine collection container assembly of FIG. 6A.

FIG. 7A is an isometric side view of a urine collection container assembly, according to an embodiment.

FIGS. 7B-7D are partial cross-sectional views of the urine collection container assembly of FIG. 7A.

FIG. 8A is an isometric view of a urine collection container assembly, according to an embodiment.

FIG. 8B is a partial cross-sectional view of the urine collection container assembly of FIG. 8A.

FIGS. 8C-8H are views of the urine collection assembly of FIG. 8A.

FIG. 9A is an exploded view of a urine collection container assembly, according to an embodiment.

FIG. 9B is a side isometric view of the urine collection container assembly of FIG. 9A.

FIGS. 9C-9D are partial side view of the urine collection container assembly of FIG. 9B.

### DETAILED DESCRIPTION

Embodiments disclosed herein are related to urine collection container assemblies, and related systems for use and methods of formation. The devices and systems disclosed herein are configured to collect fluids from an individual. The fluids collected in the fluid collection container assemblies may include at least one of urine, vaginal discharge, penile discharge, reproductive fluids, blood, sweat, or other bodily fluids.

At least one, some, or all embodiments of urine collection container systems described herein are configured to provide a technical effect of allowing for easier and more sanitary transporting and emptying of urine from urine collection container assemblies. Conventional urine collection container assemblies typically include a pull-off and press-fit lid that is difficult to seal for use and then unseal for removal. Some embodiments described herein include one or more handles on the canister, a spout on the canister, and/or one or more clamps securing the lid to the canister. Some embodiments include a rubber or silicone gasket or seal that compresses when the clamps are snap-fit onto a locking rib on the canister, causing an airtight seal. When the clamp is disengaged from the rib, the seal may decompress and the lid may be easily lifted off the canister. The spout on the canister of some embodiments provides a technical effect of preventing spilling and creating a path for urine to flow when the canister is drain. The spout may be extruded into the side of the canister and, when present, the rubber/silicon gasket of the lid may be movable to cover the spout so that the canister remains airtight when in use.

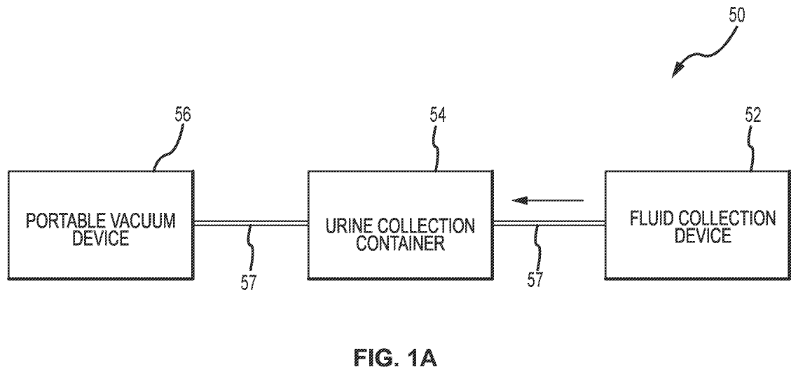

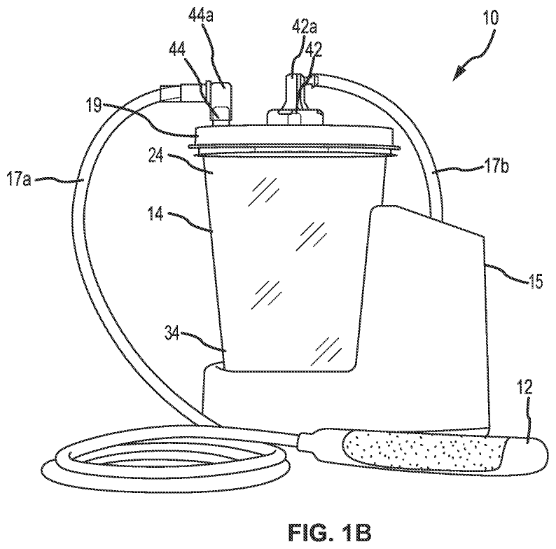

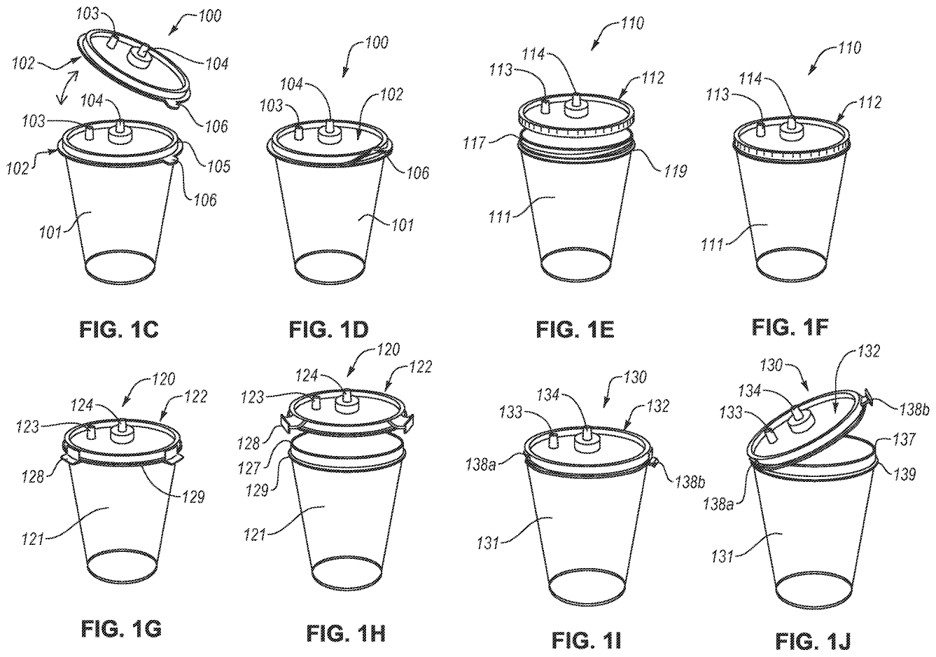

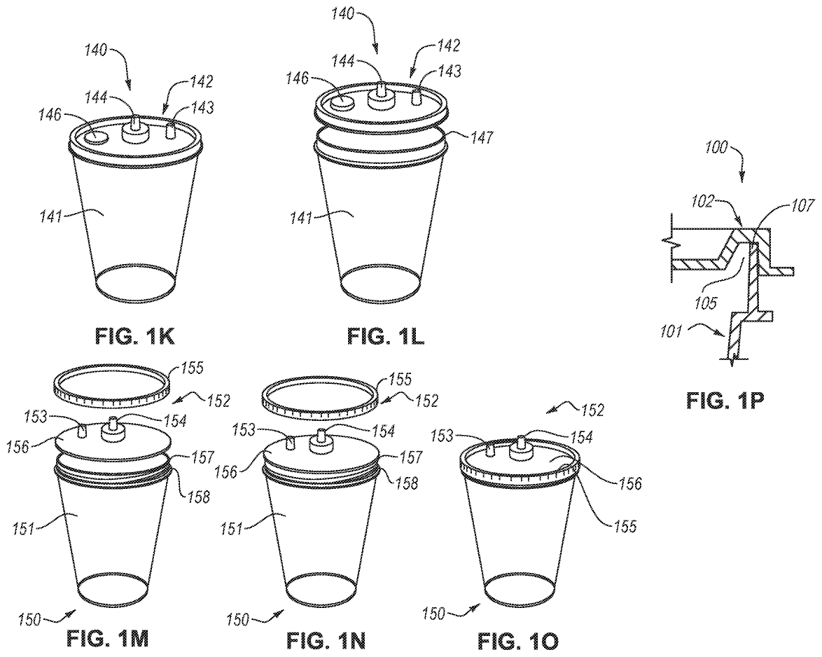

FIG. 1A is a block diagram of a fluid collection system 50, according to an embodiment. The fluid collection system 50 may be included in embodiments of fluid collection systems described herein. The system 50 includes a fluid collection device 52 (e.g., any of the fluid collection devices disclosed herein), a urine collection container assembly 54, and a pump 56 (e.g., portable vacuum device or source). The urine collection container assembly 54 may include any of the urine collection container assemblies described herein. The fluid collection device 52, the urine collection container 54, and the pump 56 may be fluidly coupled to each other via one or more conduits 57. For example, fluid collection device 52 may be operably coupled to one or more of the urine collection container 54 or the pump 56 via the conduit 57. In some embodiments, the pump 56 may be secured directly to the urine collection container 54. Fluid (e.g., urine or other bodily fluids) collected in the fluid collection device 52 may be removed from the fluid collection device 52 via the conduit 57 secured to the fluid collection device 52. Suction force may be introduced into the chamber of the fluid collection device 52 via the inlet of the conduit 57 responsive to suction (e.g., vacuum) force applied at the outlet of the conduit 57.

The suction force may be applied to the outlet of the conduit 57 by the pump 56 either directly or indirectly. The suction force may be applied indirectly via the urine collection container 54. For example, the outlet of the conduit 57 may be disposed within or fluidly coupled to an interior region of the urine collection container 54 and an additional conduit 57 may extend from the urine collection container 54 to the pump 56. Accordingly, the pump 56 may apply suction to the fluid collection device 52 via the urine collection container 54. The suction force may be applied directly via the pump 56. For example, the outlet of the conduit 57 may be disposed within the pump 56. An additional conduit 57 may extend from the pump 56 to a point outside of the fluid collection device 52, such as to the urine collection container 54. In such examples, the pump 56 may be disposed between the fluid collection device 52 and the urine collection container 54.

The urine collection container 54 is sized and shaped to retain a fluid therein. The urine collection container 54 may include a bag (e.g., drainage bag), a bottle, a canister, or a cup (e.g., collection jar), or any other enclosed container for storing bodily fluid(s) such as urine. In some examples, the conduit 57 may extend from the fluid collection device 52 and attach to the urine collection container 54 at a first point therein. An additional conduit 57 may attach to the urine collection container 54 at a second point thereon and may extend and attach to the pump 56. Accordingly, a vacuum (e.g., suction) may be drawn through fluid collection device 52 via the urine collection container 54. Fluid, such as urine, may be drained from the fluid collection device 52 using the pump 56.

The pump 56 may include one or more of a manual vacuum pump, and electric vacuum pump, a diaphragm pump, a centrifugal pump, a displacement pump, a magnetically driven pump, a peristaltic pump, or any pump configured to produce a vacuum or suction force. The pump 56 may provide a vacuum or suction to remove fluid from the fluid collection device 52. In some examples, the pump 56 may be powered by one or more of a power cord (e.g., connected to a power socket), one or more batteries, or even manual power (e.g., a hand operated vacuum pump). In some examples, the pump 56 may be sized and shaped to fit outside of, on, or within the fluid collection device 52. For example, the pump 56 may include one or more miniaturized pumps or one or more micro pumps. The vacuum sources disclosed herein may include one or more of a switch, a button, a plug, a remote, or any other device suitable to activate the pump 56.

FIG. 1B shows an example of a urine collection system 10, according to an embodiment. The urine collection system 10 may include a urine collection device 12, a first conduit 17a in fluid communication with an interior region of the urine collection device 12, a urine collection container, and a base 15 having a pump (not visible) positioned thereon or secured therein (e.g., at least partially positioned within a housing of the base 15). The urine collection container may include a canister 14 and a lid 19 selectively secured or securable to the canister 14. Although shown with the canister 14 and a lid 19 described herein, the system 10 may be used with any canister and/or lid described herein. Accordingly, any canister and/or lid described herein may be used with a system including the urine collection device 12, the first conduit 17a, second conduit 17b, and/or the base 15.

The canister 14 may include a top region 24 and a bottom region 34, and may at least partially define an interior region in fluid communication with the internal region of the urine collection device 12 via the first conduit 17a. The canister 14 may be reusable and dishwasher safe, and may include a generally rigid material such as polycarbonate or glass. In some embodiments, the canister 14 may be disposable. The urine collection system 10 may be configured to accommodate different volumetric sizes of canisters 14. The canister 14 may be substantially cylindrical or frustoconical. In some embodiments, the canister may include other shapes and configurations, such as a generally square or rectangular side profile.

The urine collection device 12 may be configured to be positioned at least proximate to a urethra of a user. While the urine collection device 12 shown in FIG. 1B includes a female urine collection device, the urine collection device 12 may instead include a male urine collection device. PCT International Application No. PCT/US2019/029616, for example, describes various embodiments of both male and female fluid collection devices, the disclosure of which are incorporated in their entirety by this reference. Moreover, the urine collection device 12 may be interchangeable in the urine collection system 10 between different types, varieties, and sizes of male or female urine collection devices. Generally, the urine collection device 12 may include a surface sized to be positioned proximate or adjacent to the urethra and configured to wick urine or other fluids away from the user. Urine or other fluids may be wicked from the surface to a reservoir in the urine collection device 12.

The urine collection system 10 also includes the first conduit 17a in fluid communication with an interior region (e.g. reservoir) of the urine collection device 12 and the interior region of the canister 14. The first conduit 17a may be positioned between the urine collection device 12 and the canister 14. The urine collection system 10 also may include the second conduit 17b providing fluid communication between the pump 16 and the interior region of the canister 14. In some embodiments, the pump may be secured directly to the canister 14, and the second conduit 17b may be absent from the urine collection system 10. The conduits 17a, 17b may include a flexible tube. In some embodiments, at least a portion of the first conduit 17a is substantially opaque, thereby inhibiting viewing of the urine within the first conduit 17a.

The pump is in fluid communication with the interior region of the canister 14 and is configured to pull a vacuum on the interior region of the canister 14 effective to draw the urine from the urine collection device 12 through the first conduit 17a into the canister 14. The pump 16 may be secured directly to the canister 14, or the conduit 17b may fluidly couple the pump 16 with the interior region of the canister 14. The pump 16 may include one or more of a manual vacuum pump, and electric vacuum pump, a diaphragm pump, a centrifugal pump, a displacement pump, a magnetically driven pump, a peristaltic pump, or any pump configured to produce a vacuum. The pump 16 may provide a vacuum or suction to remove fluid from the fluid collection device 12. In some examples, the pump 16 may be powered by one or more batteries or other power sources. The pump 16 may be included in a housing that also supports the canister 14. In some examples, the pump 16 may be sized and shaped to fit within a container support on a wheelchair. In some embodiments, the pump 16 may include a wall-mounted pump. The pump 16 may include one or more of a switch, a button, a plug, a remote, or any other device suitable to activate the pump 16.

The lid 19 may include multiple ports 42, 44 for attachment of the conduits 17a, 17b thereto. One or more of the multiple ports 42, 44 may include an adapter (e.g., adapters 42a, 44a) detachably secured or securable thereto that improves attachments of the conduits 117a, 117b to the multiple ports 42, 44. In some embodiments, the lid 19 may include additional or fewer ports or adapters than those shown in FIG. 1B. Except for the ports and/or adapters for attachment of the conduits 17a, 17b, the canister 14 may be sealed and airtight such that the urine collected in the canister 14 does not leak or spill.

In some embodiments, the lid 19 may include a urine port 44 to which an outlet of the first conduit 17a may be detachably secured (either directly or via an adapter 44a secured to the urine port 44) to provide fluid communication between urine collection device 12 and the interior region of the canister 14. A second conduit 17b may provide fluid communication between the interior region of the canister 14 and the pump. For example, the lid 19 may include a vacuum port 42 to which an outlet of the second conduit 17b may be detachably secured (either directly or via an adapter 42a secured to the vacuum port 42) to provide fluid communication between the pump and the interior region of the canister. The vacuum port 42 and/or the urine port 44 may include adapters 42a, 44a, respectively, detachably secured thereto to secure the respective conduit 17b, 17a to the vacuum port 42 and/or the urine port 44. In some embodiments, at least one (e.g., both) of the first conduit 17a and the second conduit 17b may be detachably connected or secured to the canister 14 effective to provide fluid communication with the interior region of the canister 14. For example, the canister may include at least one (e.g. both) of the urine port 44 and the vacuum port 42. When the lid 19 is secured to the canister 14, an inlet of the first conduit 17a may be secured to the urine collection device 12, the outlet of first conduit may be secured to the urine port 44, the second conduit 17b may be secured to the vacuum port 42, and activation of the pump may create a negative pressure in the interior region of the canister 14 effective to draw urine in the urine collection device 12 through the first conduit 17a into the interior region of the canister 14.

FIGS. 1C-1O are isometric side views of various embodiments of urine collection container assemblies including a canister and a lid. The various embodiments illustrated in FIGS. 1C-1O are configured to improve upon conventional urine collection container assemblies having lids that snap- or press-fit to the top of the canister. Each of the urine collection container assembly embodiments illustrated in FIGS. 1C-1O are configured to allow a lid to be selectively secured or mounted to and removed from the canister. Each of the lid embodiments include the two ports described above. Unless otherwise noted, each of the urine collection container assembly embodiments illustrated in FIGS. 1C-1O may include any aspect of the urine collection container assembly 10.

Turning specifically to FIGS. 1C-1D a urine collection container assembly 100 may include a canister 101 and a lid 102 configured to selectively secure to the canister 101. The lid 102 may include multiple ports 103, 104 for attachment of the conduits 17a, 17b thereto. Except for the ports and/or adapters for attachment of the conduits 17a, 17b, the lid 102 may be sealed substantially airtight with the canister 101 such that the urine collected in the canister 101 does not leak or spill.

In many embodiments, the lid 102 is configured to be pressed onto an upper edge (e.g., rim) of the canister 101. For example, the lid 102 may include one or more walls defining a slot 105 (shown in FIG. 1P) sized and dimensioned to seal around the upper edge of the canister 101. FIG. 1P shows a partial cross-sectional view of an upper edge 107 of the canister 101 disposed within the slot 105 defined by one or more walls of the lid 102. In some embodiments, the slot 105 is sized such that the upper edge 107 of the canister 101 interfaces all of the one or more walls of the lid 102 defining the slot 105. The lids of any urine collection container assemblies described herein may include a similarly configured slot 105. The press-on lid 102 may include a more flexible or deformable material, such as silicone. Returning to FIGS. 1C-1D, the lid 102 also may include a tab 106 configured to allow a user to pull off the lid 102 from the canister 102.

Turning specifically to FIGS. 1E-1F a urine collection container assembly 110 may include a canister 111 and a lid 112 configured to selectively secure to the canister 111. The lid 112 may include multiple ports 113, 114 for attachment of the conduits 17a, 17b thereto. Except for the ports and/or adapters for attachment of the conduits 17a, 17b, the lid 112 may be sealed substantially airtight with the canister 111 such that the urine collected in the canister 111 does not leak or spill.

In many embodiments, the lid 112 is configured to threadedly secure to the canister 111. Both the lid 112 and the canister 111 may include complementary threads for threaded engagement. For example, the canister 111 may include threads 117 proximate to the upper edge (e.g., rim) of the canister 111, and the lid 112 may include threads (not visible) within a slot defined by one or more walls of the lid 112. The canister 111 also may include a flange 119 disposed on the canister 111 proximate to the upper edge with the threads 117 disposed between the flange 119 and the upper edge of the canister 111.

Turning specifically to FIGS. 1G-1H a urine collection container assembly 120 may include a canister 121 and a lid 122 configured to selectively secure to the canister 121. The lid 122 may include multiple ports 123, 124 for attachment of the conduits 17a, 17b thereto. Except for the ports and/or adapters for attachment of the conduits 17a, 17b, the lid 122 may be sealed substantially airtight with the canister 121 such that the urine collected in the canister 121 does not leak or spill.

In many embodiments, the lid 122 includes multiple clamps 128 configured to selectively clip, lock, or otherwise engage with a flange 129 (e.g., lip) proximate to the upper edge 127 of the canister 121. For example, the upper edge region of the canister 121 may be inserted into a slot defined by one or more walls of the lid 122, and each of the multiple clamps 128 may be adjusted or folded to engage with the flange 129 to secure the lid 122 to the canister 121. Each of the multiple clamps 128 may be pivotably secured to the lid 122 or flexible and formed integrally with the lid 122 to allow a user to selectively engage and disengage the multiple clamps 128 from the flange 129 multiple times.

Turning specifically to FIGS. 1I-1J a urine collection container assembly 130 may include a canister 131 and a lid 132 configured to selectively secure to the canister 131. The lid 132 may include multiple ports 133, 134 for attachment of the conduits 17a, 17b thereto. Except for the ports and/or adapters for attachment of the conduits 17a, 17b, the lid 132 may be sealed substantially airtight with the canister 131 such that the urine collected in the canister 131 does not leak or spill.

In many embodiments, the lid 132 is hingedly secured to the canister 131. For example, the lid 132 may include a hinge 138 hingedly securing the lid 131 to the canister 131 and a clamp 138b configured to selectively clip, lock, or otherwise engage with a flange 139 (e.g., lip) proximate to the upper edge 137 of the canister 131. For example, the upper edge region of the canister 131 may be inserted into a slot defined by one or more walls of the lid 132, and the clamp 138b may be adjusted or folded to engage with the flange 139 to secure the lid 132 to the canister 131. The clamp 138 may be pivotably secured to the lid 132 or flexible and formed integrally with the lid 132 to allow a user to selectively engage and disengage the clamp 138 from the flange 139 multiple times.

Turning specifically to FIGS. 1K-1L a urine collection container assembly 140 may include a canister 141 and a lid 142 configured to selectively secure to the canister 141. The lid 142 may include multiple ports 143, 144 for attachment of the conduits 17a, 17b thereto. Except for the ports and/or adapters for attachment of the conduits 17a, 17b, the lid 142 may be sealed substantially airtight with the canister 141 such that the urine collected in the canister 141 does not leak or spill.

In many embodiments, the lid 142 includes a push button 146 that, when pressed while the lid 142 is secured to the canister 141, increases pressure within the interior region of the canister 142, thereby forcing the lid 142 off the canister 141 or allowing the lid 142 to be more easily removed from the canister 141. In some embodiments, when the push button 146 is pressed while the lid 142 is secured to the canister 141, the lid 142 is locked in place on the canister 141. In these and other embodiments, the push button 146 may be pressed again to release a locking mechanism or increases pressure within the interior region of the canister 142 for removal of the lid. The lid 142 may include a slot sized and dimensioned to seal around the upper edge 147 of the canister 142.

Turning specifically to FIGS. 1M-1O a urine collection container assembly 150 may include a canister 151 and a lid 152 configured to selectively secure to the canister 151. The lid 151 may include multiple ports 153, 154 for attachment of the conduits 17a, 17b thereto. Except for the ports and/or adapters for attachment of the conduits 17a, 17b, the lid 152 may be sealed substantially airtight with the canister 151 such that the urine collected in the canister 151 does not leak or spill.

The lid 151 may include a screw band 155 and substantially flat cover 156 that includes the ports 153, 154. The cover 156 may be placed on the upper edge 157 of the canister 151 and then the screw band 155 may be threadedly engaged with threads 158 on the canister 151 proximate to the upper edge 157 to seal the cover 156 to the lid 151 (shown in FIG. 1O).

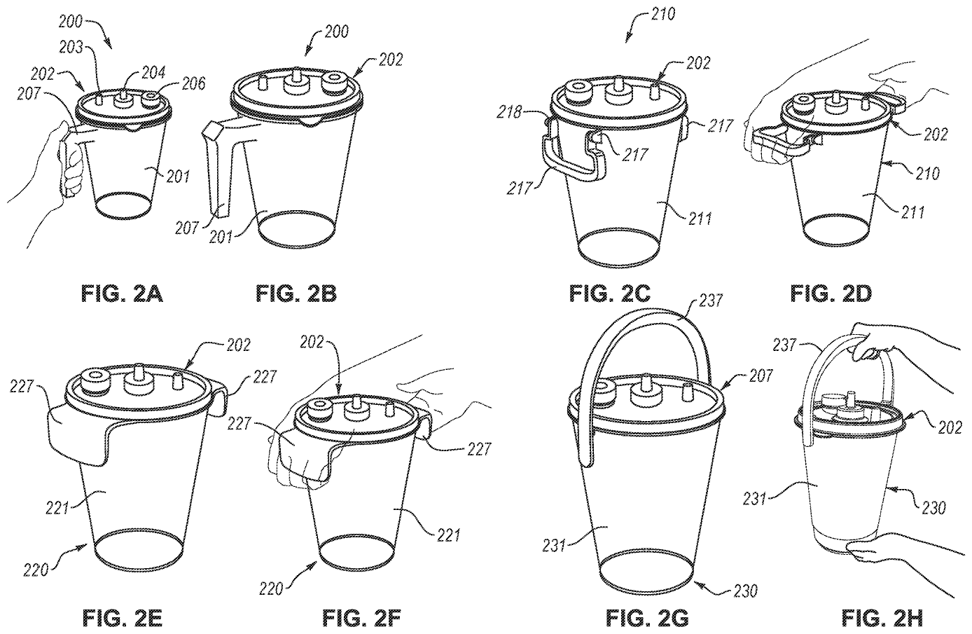

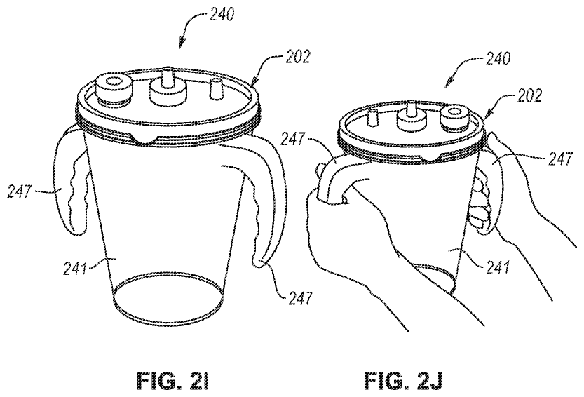

FIGS. 2A-2J are isometric side views of various embodiments of urine collection container assemblies including a canister having one or more handles and lid. The various embodiments illustrated in FIGS. 2A-2J are configured to allow a user to more easily carry, move, and/or empty the canister. Each of the urine collection container assembly embodiments illustrated in FIGS. 2A-2J is configured to allow a lid to be selectively secured or mounted to and removed from the canister. Each of the lid embodiments include the two ports described above. Unless otherwise noted, each of the urine collection container assembly embodiments illustrated in FIGS. 2A-2J may include any aspect of the urine collection container assemblies 10, 100, 110, 120, 130, 140, 150. For example, any embodiments of urine collection container assemblies illustrated in FIGS. 2A-2J may include any of the lids 102, 112, 122, 132, 142, 152 and corresponding features in the containers 101, 111, 121, 131, 141, 151 that allow for securement and/or sealing between the lids and containers.

Turning specifically to FIGS. 2A-2B a urine collection container assembly 200 may include a canister 201 and a lid 202 configured to selectively secure or mount to the canister 201. The lid 202 may include multiple ports 203, 204 for attachment of the conduits 17a, 17b thereto. The lid 202 also may include a selectively openable and closeable spout 206. The spout 206 may include a cap threadedly and selectively coupled to a port on the lid 202. In some embodiments, the spout 206 is absent. Except for spout 206, the ports and/or adapters for attachment of the conduits 17a, 17b, the lid 102 may be sealed substantially airtight with the canister 201 such that the urine collected in the canister 201 does not leak or spill. The spout 206 may be closed to complete the seal of the urine collection container assembly 200 when urine is being drawing from a urine collection device. In some embodiments, the canister 201 may include a pitcher style handle, such as one or more L- or C-shaped handles 207 extending from the sidewall of the canister 201.

Turning to FIGS. 2C-2D, in some embodiments, a urine collection container assembly 210 may include the lid 202 and a canister 211 having two opposing handles 217 hingedly or pivotably secured to the canister 211. For example, the canister 211 may include hinge members 218 secured to an upper region of the canister 211, and each of the handles 217 may be hingedly or pivotably secured to two hinge members 218. When released by a user, the handles 217 may fall or swing towards the canister 211. When grasped by a user, the handles 217 may swing to be generally parallel with the top of the canister. A flange on the canister 211 or a stop element on at least one the hinge members 218 may prevent the handles 217 from swinging past the plant of the top of the canister 211.

Turning to FIGS. 2E-2F, in some embodiments, a urine collection container assembly 220 may include the lid 202 and a canister 221 having two opposing handles 227 extending from the canister 221 proximate to the top of the canister 227. The handles 227 may be formed integrally with the canister 221 and may be generally rigid. In some embodiments, the handles 227 may extend from the flange at the upper region of the canister 221.

Turning to FIGS. 2G-2H, in some embodiments, a urine collection container assembly 230 may include the lid 202 and a canister 231 having a handle 237 hingedly or pivotably secured to the canister 231. For example, the handle 237 may include a bucket-type handle hingedly or pivotably secured to the canister 231 at two opposing regions of the canister 231. The handle 237 may swing or pivot over the lid 202 when the lid is secured to the canister 231. In some embodiments, the handle 237 may include one or more tabs configured to lock the lid 202 to the canister 231 when the handle is in an upright position.

Turning specifically to FIGS. 2I-2J a urine collection container assembly 240 may include a pitcher style handle, such as two L- or C-shaped handles 247 extending from the sidewall of the canister 201.

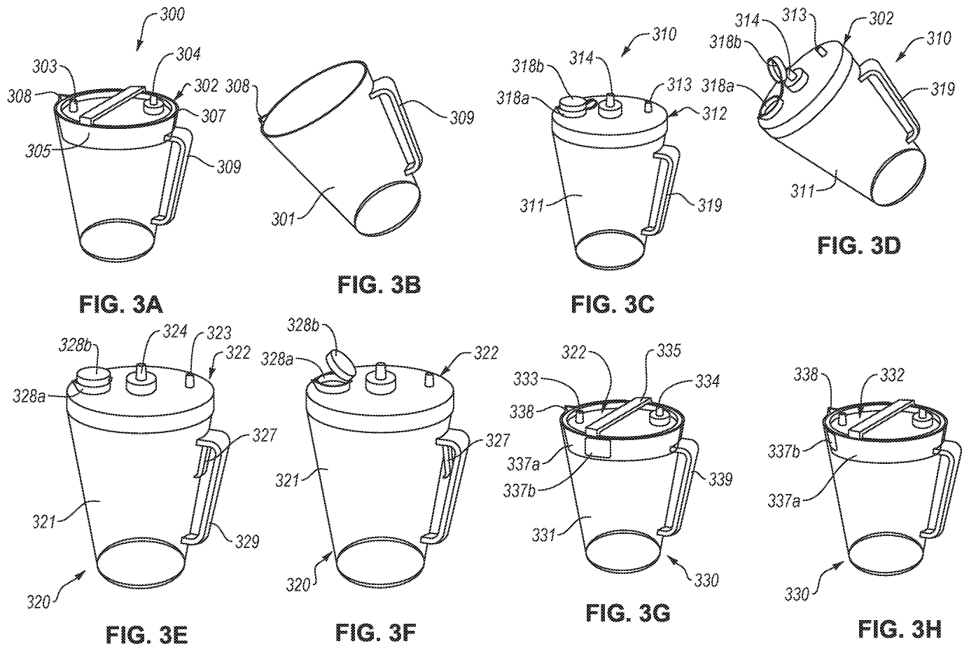

FIGS. 3A-3H are isometric side views of various embodiments of urine collection container assemblies including a canister having one or more handles, a lid, and a spout on at least one of the lid or the canister. The various embodiments illustrated in FIGS. 3A-3H are configured to allow a user to more easily pour or empty the canister. Each of the lid embodiments include the two ports described above. Unless otherwise noted, each of the urine collection container assembly embodiments illustrated in FIGS. 2A-2J may include any aspect of the urine collection container assemblies 10, 100, 110, 120, 130, 140, 150, 200, 210, 220, 230, 240.

Turning specifically to FIGS. 3A-3B, a urine collection container assembly 300 may include a canister 301 and a lid 302 configured to selectively secure or mount to the canister 301. The lid 302 may include multiple ports 303, 304 for attachment of the conduits 17a, 17b thereto. Except for the ports and/or adapters for attachment of the conduits 17a, 17b, the lid 302 may be sealed substantially airtight with the canister 301 such that the urine collected in the canister 301 does not leak or spill. The canister 301 may include a handle 309 and a generally frustoconical shape that narrows from the upper edge 307 of the canister 301 to the bottom.

The canister also may include a spout 308 protruding from the canister 301 at least proximate to the upper edge 307 of the canister 301. The spout 308 may be generally opposite or distal to the handle 309. The lid 302 also may include rim 305 that covers the spout 308 such that the container assembly 300 is substantially sealed and airtight when the lid 302 is secured to the canister 301 (shown in FIG. 3A). The rim 305 may narrow complementary to a portion of the canister 301 such that the lid 302 nests at least partially within the canister 301 when the lid 302 is secured to or mounted on the canister 301. The lid 302 also may include a handle to allow for more easy removal for the lid 302 for emptying of the canister 301. FIG. 3B illustrates the canister 301 without the lid 302 when the canister 301 may be emptied or cleaned.

Turning specifically to FIGS. 3C-3D, a urine collection container assembly 310 may include a canister 311 and a lid 312 configured to selectively secure or mount to the canister 311. The lid 312 may include multiple ports 313, 314 for attachment of the conduits 17a, 17b thereto. Except for the ports and/or adapters for attachment of the conduits 17a, 17b, the lid 312 may be sealed substantially airtight with the canister 311 such that the urine collected in the canister 311 does not leak or spill. The canister 311 may include a handle 319 and a generally frustoconical shape that narrows between the upper edge of the canister 311 to the bottom of the canister. In some embodiments, the canister 311 includes a generally cylindrical portion proximate the upper edge of the canister 311, and the lid 312 may be sized complementary to the cylindrical portion such that lid 311 may rest within the cylindrical portion or the cylindrical portion may be inserted into an interior region of the lid 312 to secure or mount the lid 312 and the canister 311 together.

The lid 312 also may include a spout 318a and a cap 318b configured to selectively and removably secure to and cover the spout 318a. With the cap 318b secured to and covering the spout 318a (shown in FIG. 3C), the container assembly 310 is substantially sealed and airtight for removal of urine from a urine collection device. FIG. 3D illustrates the cap 318b removed from the spout 318a when the canister 311 may be emptied.

Turning specifically to FIGS. 3E-3E, a urine collection container assembly 320 may include a canister 321 and a lid 322 configured to selectively secure or mount to the canister 321. The lid 322 may include multiple ports 323, 324 for attachment of the conduits 17a, 17b thereto. Except for the ports and/or adapters for attachment of the conduits 17a, 17b, the lid 322 may be sealed substantially airtight with the canister 321 such that the urine collected in the canister 321 does not leak or spill. The canister 321 may include a handle 329 and a generally frustoconical shape that narrows between the upper edge of the canister 321 to the bottom of the canister. In some embodiments, the canister 321 includes a generally cylindrical portion proximate the upper edge of the canister 321, and the lid 322 may be sized complementary to the cylindrical portion such that lid 321 may rest within the cylindrical portion or the cylindrical portion may be inserted into an interior region of the lid 322 to secure or mount the lid 322 and the canister 321 together.

The lid 322 also may include a spout 328a and a cap 328b configured to selectively and removably secure to and cover the spout 328a. The urine collection container assembly 320 also may include an actuator 327 (e.g., lever or trigger) disposed at least proximate to the handle 329 of the canister 321. The actuator 327 may be cooperatively coupled to the cap 328b such that pulling the actuator 327 opens the spout 328a and releasing the actuator 327 closes the spout 328a. With the cap 328b secured to and covering the spout 328a (shown in FIG. 3E), the container assembly 320 is substantially sealed and airtight for removal of urine from a urine collection device. FIG. 3F illustrates the cap 328b removed from the spout 328a when the canister 321 may be emptied.

Turning specifically to FIGS. 3G-3H, a urine collection container assembly 330 may include a canister 331 and a lid 332 configured to selectively secure or mount to the canister 331. The lid 332 may include multiple ports 333, 334 for attachment of the conduits 17a, 17b thereto. Except for the ports and/or adapters for attachment of the conduits 17a, 17b, the lid 332 may be sealed substantially airtight with the canister 331 such that the urine collected in the canister 331 does not leak or spill. The canister 331 may include a handle 339 and a generally frustoconical shape that narrows from the upper edge of the canister 331 to the bottom.

The canister also may include a spout 338 protruding from the canister 331 at least proximate to the upper edge of the canister 331. The spout 338 may be generally opposite or distal to the handle 339. The lid 332 also may include rim 337a that covers the spout 338 such that the container assembly 330 is substantially sealed and airtight when the lid 332 is secured or mounted to the canister 331 in a closed position (shown in FIG. 3G). The rim 337a also may define an opening 337b. The lid 332 is rotatable when mounted or secured to the canister 331, and the lid 332 may be rotated to an open position in which the opening 337b is aligned with the spout 338 for emptying of the canister 331. The rim 337a may narrow complementary to a portion of the canister 331 such that the lid 332 nests at least partially within the canister 331 when the lid 332 is secured to or mounted on the canister 331. The lid 332 also may include a handle 335 to allow for more easy removal and/or rotation of the lid 332.

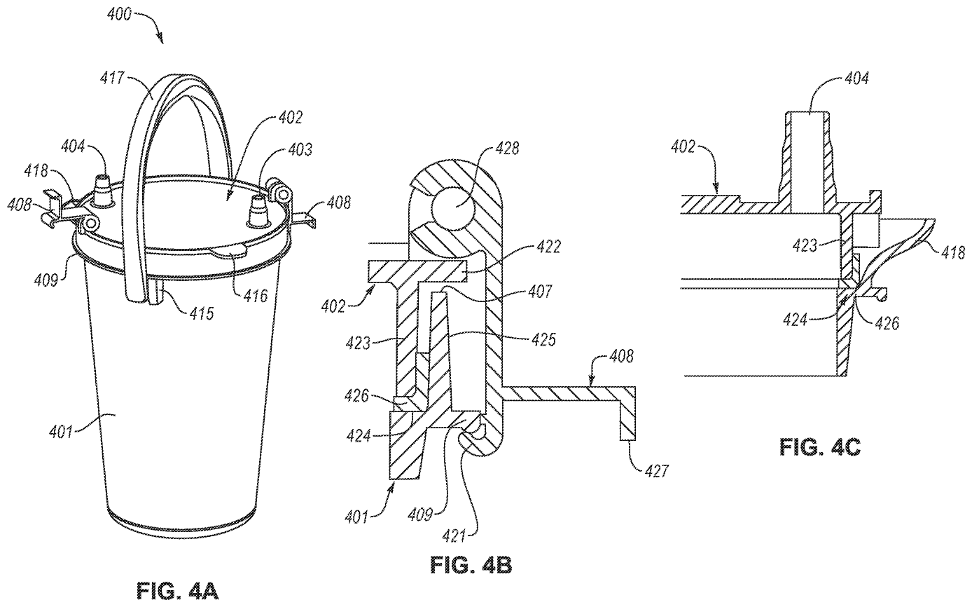

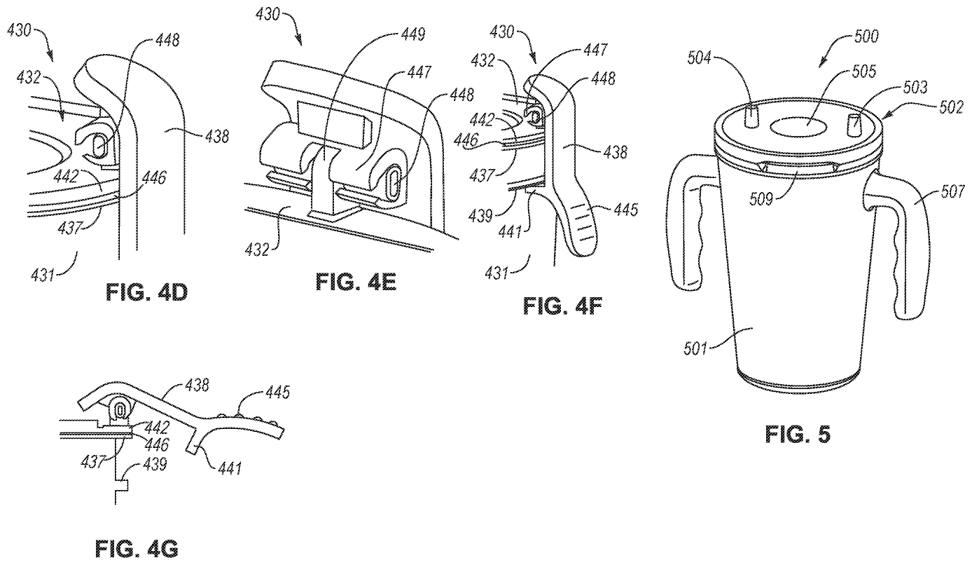

Turning now to FIGS. 4A-4C, a urine collection container assembly 400 may include a canister 401 and a lid 402 configured to selectively secure or mount to the canister 401. The lid 402 may include multiple ports 403, 404 for attachment of the conduits 17a, 17b thereto. Except for the ports and/or adapters for attachment of the conduits 17a, 17b, the lid 402 may be sealed substantially airtight with the canister 401 such that the urine collected in the canister 401 does not leak or spill.

The canister 401 may include an outer surface, an interior region, an upper region 425 having an upper edge 407, and a locking rib 409 on the outer surface of the canister 401 at least proximate to the upper region 425 of the canister 401. In some embodiments, the canister 401 includes a shelf 424 spaced from the upper edge 407 of the canister 401 and projecting inward. In some embodiments, the upper region 425 may extend upward between the locking rib 409 and the shelf 424. The canister 401 also may include a spout 418 protruding from the canister 401 at least proximate to the upper edge 407 of the canister 401. The urine collection container assembly 400 also may include at least one handle on the canister 401. For example, the urine collection container assembly 400 may include a handle 417 having two opposing end regions hingedly secured to opposing portions 415 of an upper portion of the canister.

The lid 402 is secured or securable to the canister 401 and may include a flange 422 forming an outer periphery of the lid 402 that extends outward beyond the upper edge 407 of the canister when the lid 402 is secured or mounted to the canister 401. The lid 402 also may include a rim 423 spaced inward from the outer periphery of the flange 422. In some embodiments, the lid 402 includes one or more (e.g., two) clamp supports 428 secured thereto or formed integrally therewith. The clamp supports 428 may extend upward, generally opposite to the canister 401 with the lid 402 is secured to the canister 401. The lid 402 also may include a tab 416 extending from the outer periphery of the lid 402 and sized and dimensioned to allow a user to grip the tab 416 to remove the lid 402.

The urine collection container assembly 400 also includes one or more (e.g., two) clamps 408 pivotably or hingedly secured to the lid 402. For example, the clamp 408 may be pivotably or hingedly secured to the clamp support 428. The clamp 408 may include an inward oriented projection 421 configured to selectively engage with at least a portion of the locking rib 409 to secure the lid 402 to the canister 401. Each clamp 421 may be movable between an unlocked position (shown in FIG. 4A) in which the inward oriented projection 421 is disengaged from the locking rib 409, and a locked position (shown in FIG. 4B) in which the inward oriented projection 421 is engaged with the locking tab 409. In some embodiments, each of the one or more clamps 408 may include a finger grip 427 extending outward from the clamp 408 and configured to allow a user to more easily engage and disengage the clamp 408 from the locking rib 409.

The urine collection container assembly 400 also may include a seal or gasket positioned and configured to form a seal between the lid 401 and the canister 402 when the one or more clamps 408 are engaged with the locking rib 409. In some embodiments, a gasket 426 is disposed between the rim 423 and the shelf 426 of the canister 401 when the lid 402 is secured to the canister 401 with the rim 423 disposed at least partially within the canister 401. The gasket 426 also may disposed between the rim 423 and the upper region 425 of the canister 401 in addition or alternative to being disposed between the rim 423 and the shelf 426 of the canister 401. Accordingly, in some embodiments, the gasket 426 is L-shaped, and may rest on or interface with the shelf 424 and an inner surface of the upper region 425 of the canister 401. In some embodiments, the gasket 446 also may disposed between the rim 423 and the upper region 425 of the canister 401 in addition or alternative to being disposed between the upper edge 407 of the canister 401 and the flange 422 of the lid 402. Accordingly, in some embodiments, the gasket 426 is L-shaped, and may rest on or interface with the flange 422 and an inner surface of the rim 423 of the lid 402. As illustrated in FIG. 4C, the gasket 426 may form a seal that prevents fluid communication between the spout 418 and the interior region of the canister 401 when the lid 402 is secured to the canister 401.

In some embodiments, clamps may be similar used and disposed to secure or mount a lid to a canister, but the gasket may be positioned elsewhere between the lid and the canister. Turning specifically to FIGS. 4D-4G, portions of a urine collection container assembly 430 are shown. Unless otherwise note, the urine collection container assembly 430 may include any aspect of the urine collection container assembly 400. In some embodiments, the urine collection canister assembly 430 includes a canister 431 having an outer surface, an interior region, an upper region having an upper flanged edge 437, and a locking rib 439 on the outer surface of the canister at least proximate to the upper region of the canister 431.

The urine collection container assembly 430 may include a lid 432 secured or securable (e.g., mounted or mountable) to the canister 431. In many embodiments, the lid 432 includes a vacuum port (not visible) configured to detachably secure to a first conduit to provide fluid communication between a pump and the interior region of the canister 431, a urine port (not visible) configured to detachably secure to a second conduit to provide fluid communication between a urine collection device and the interior region of the canister 431, and at least one clamp 438 having an inward oriented projection 441 configured to selectively engage with at least a portion of the locking rib 439 to secure the lid 432 to the canister 431. While only one clamp 438 is visible in FIGS. 4D-4G, many embodiments of the urine collection container assembly 430 may include two opposing clamps 438, similar to the positioning of the clamps 408 in the urine collection container assembly 400. The lid 432 may include an outer periphery 442 having a diameter substantially equal to the diameter of the flanged upper edge 437 of the canister 431.

The lid 432 may include a clamp support post 449 having arms 448 extending therefrom, and the clamp 438 may be configured to detachably secure to the arms 448 to allow pivoting of the clamp 438 when secured to the arms 448. For example, the clamp 438 may include a C-shaped member 447 that snap-fits to the arm 448 to allow pivoting of the clamp 438 from a locked position (shown in FIG. 4F) having the inward oriented projection 441 engaged with the locking rib 439 and an unlocked position (shown in FIG. 4G) having the inward oriented projection disengaged from the locking rib 439. The clamp 438 also may include one or more finger grips 445 disposed generally distal to the C-shaped member 447.

The urine collection container assembly 430 also may include a gasket 446 or seal. In some embodiments, the gasket 446 may be disposed between the upper flanged edge 437 of the canister 432 and the lid 432 when the lid 432 is secured to the canister 431. In these and other embodiments, the rim may be absent from the lid 432.

FIG. 5 illustrates a side isometric view of another urine collection container assembly 500 including a canister 501 and a lid 502 detachably and selectively secured to the canister 501 to form an airtight seal. The canister 501 may include one or more L- or C-shaped handles 507, and the lid 502 may include multiple ports 503, 504 for attachment of the conduits 17a, 17b thereto. The lid 502 also may include a button 505 on the top thereof and a silicone gasket or seal (not visible) that interfaces an upper region of the canister 501 when the lid 502 is at least partially inserted into the canister 501. The gasket may be cooperatively coupled or cooperatively connected to the button 505 such that when the button 505 is pressed, the gasket is pushed or forced outward against the inner surface of the canister 501, thereby forming an airtight seal. The button 505 may be pressed a second time to contract the gasket and release the airtight seal, allowing for more easy removal of the lid 502. The lid 502 also may include recessed finger grips 509. In some embodiments, the urine collection container assembly 500 may include a cam-style lid 502 having a cam and groove-type fitting with the canister 501.

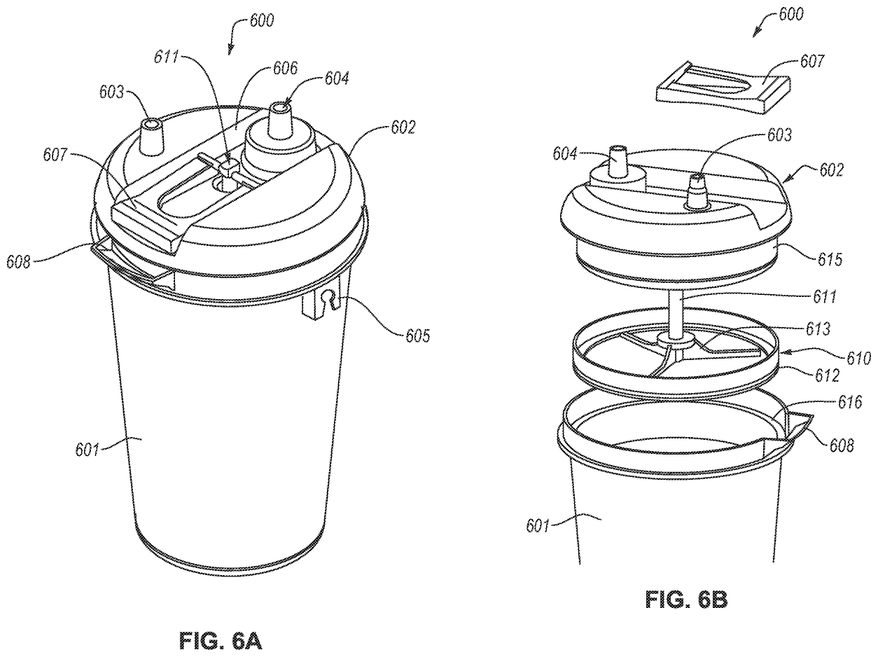

Turning now to FIG. 6A-6B, in some embodiments, a urine collection container assembly 600 may include a lever-type assembly configured to secure or mount a lid 602 to a canister 601. Similar to other embodiments, the canister may include a spout 608 and a hinge member 605 configured to detachably secure a handle (not shown) to the canister 601. The lid 602 may include multiple ports 603, 604 for attachment of the conduits 17a, 17b thereto.

The urine collection container assembly 600 also may include a seal assembly 610 positioned within the canister 601 and operably connected to a movable lever 607. For example, the seal assembly 610 may include a base including a seal 612 or gasket, a post 611, and one or more arms extending between and/or connective the post 611 with the base. The lid 602 may include an opening through which the post 611 extends. The opening may be sized to allow movement of the post 611 through the opening, and also may include a seal between the portion of the lid 602 defining the opening and the post 611.

The lever 607 may be detachably and pivotably secured or securable to an end region of the post 611 distal to the base including the seal 612. For example, the post 611 may include a hook configured to snap-fit or press-fit to a bar of the lever 607. The when the urine collection container assembly 600 is assembled, the lever 607 is movable between a first or sealing position and a second or unsealed position. When the lever is in the first or sealed position, the lever 607 may push or pull the seal 612 within the canister 601 to form an airtight seal within the canister 601 such that fluid communication with the interior region of the canister 601 occurs only through the ports 603, 604. When the lever 607 is in the second or unsealed position, the seal 612 is released such that fluid in the canister 601 may be poured out of the spout 608 of the canister 601 and/or the lid 602 removed from the canister 601. In some embodiments, the canister 601 includes a rib 616 or shelf configured to engage or interface with the seal 612 when the lever 607 is in the sealed position to form the airtight seal in the interior region of the canister 601.

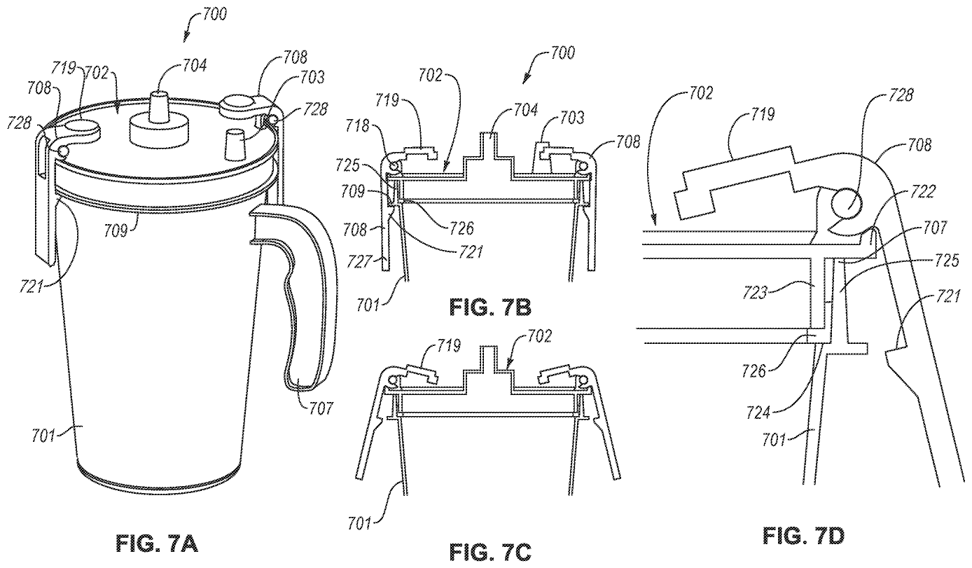

Turning now to FIGS. 7A-7C, a urine collection container assembly 700 may include a canister 701 and a lid 702 configured to selectively secure or mount to the canister 701. The lid 702 may include multiple ports 703, 704 for attachment of the conduits 17a, 17b thereto. Except for the ports and/or adapters for attachment of the conduits 17a, 17b, the lid 702 may be sealed substantially airtight with the canister 701 such that the urine collected in the canister 701 does not leak or spill.

The canister 701 may include an outer surface, an interior region, an upper region 723 having an upper edge 707, and a locking rib 709 on the outer surface of the canister 701 at least proximate to the upper region 725 of the canister 701. In some embodiments, the canister 701 includes a shelf 724 spaced from the upper edge 707 of the canister 701 and projecting inward. In some embodiments, the upper region 725 may extend upward between the locking rib 709 and the shelf 724. The canister 701 also may include a spout 718 protruding from the canister 701 at least proximate to the upper edge 707 of the canister 701. The urine collection container assembly 700 also may include at least one handle on the canister 701. For example, the urine collection container assembly 700 may include a C- or L-shaped handle 707 secured to an upper portion of the canister 701.

The lid 702 is secured or securable to the canister 701 and may include a flange 722 forming an outer periphery of the lid 702 that extends outward beyond the upper edge 707 of the canister 701 when the lid 702 is secured or mounted to the canister 701. The lid 702 also may include a rim 723 spaced inward from the outer periphery of the flange 722. In some embodiments, the lid 702 includes one or more (e.g., two) clamp supports 728 secured thereto or formed integrally therewith. The clamp supports 728 may extend upward, generally opposite to the canister 701 with the lid 702 is secured to the canister 701.

The urine collection container assembly 700 also includes one or more (e.g., two) clamps 708 pivotably or hingedly secured to the lid 702. For example, the clamp 708 may be pivotably or hingedly secured to the clamp support 728. The clamp 708 may include an inward oriented projection 721 configured to selectively engage with at least a portion of the locking rib 709 to secure the lid 702 to the canister 701. Each clamp 721 may be movable between an unlocked position (shown in FIG. 7C) in which the inward oriented projection 721 is disengaged from the locking rib 709, and a locked position (shown in FIG. 7B) in which the inward oriented projection 721 is engaged with the locking tab 709. In some embodiments, each of the one or more clamps 708 may include an elongated finger grip 727 extending past the inward oriented projection 721 such that the inward oriented projection 721 is disposed between a distal end of the finger grip 727 and the clamp support 728. The clamp 708 also may include a proximal end region 719 that extends over the lid 701. The proximal end region 719 may be sized and dimensions such that when the clamp 708 is in the open position, a portion of the proximal end region 719 may contact the top of the lid 719 to assist in breaking the seal and remove the lid 702 from the canister 701. The proximal end region 719 also may include an enlarged (e.g., circular) portion that allows a user to more easily press the proximal end region 719 to disengage the inward oriented projection 721 from the locking rib 709.

The urine collection container assembly 700 also may include a seal or gasket positioned and configured to form a seal between the lid 701 and the canister 702 when the one or more clamps 708 are engaged with the locking rib 709. In some embodiments, a gasket 726 is disposed between the rim 723 and the shelf 726 of the canister 701 when the lid 702 is secured to the canister 701 with the rim 723 disposed at least partially within the canister 701. The gasket 726 also may disposed between the rim 723 and the upper region 725 of the canister 701 in addition or alternative to being disposed between the rim 723 and the shelf 726 of the canister 701. Accordingly, in some embodiments, the gasket 726 is L-shaped, and may rest on or interface with the shelf 724 and an inner surface of the upper region 725 of the canister 701. In some embodiments, the gasket 746 also may disposed between the rim 723 and the upper region 725 of the canister 701 in addition or alternative to being disposed between the upper edge 707 of the canister 701 and the flange 722 of the lid 702. Accordingly, in some embodiments, the gasket 726 is L-shaped, and may rest on or interface with the flange 722 and an inner surface of the rim 723 of the lid 702. As illustrated in FIG. 7D, the gasket 726 may form a seal that prevents fluid communication between the spout 718 and the interior region of the canister 701 when the lid 702 is secured to the canister 701.

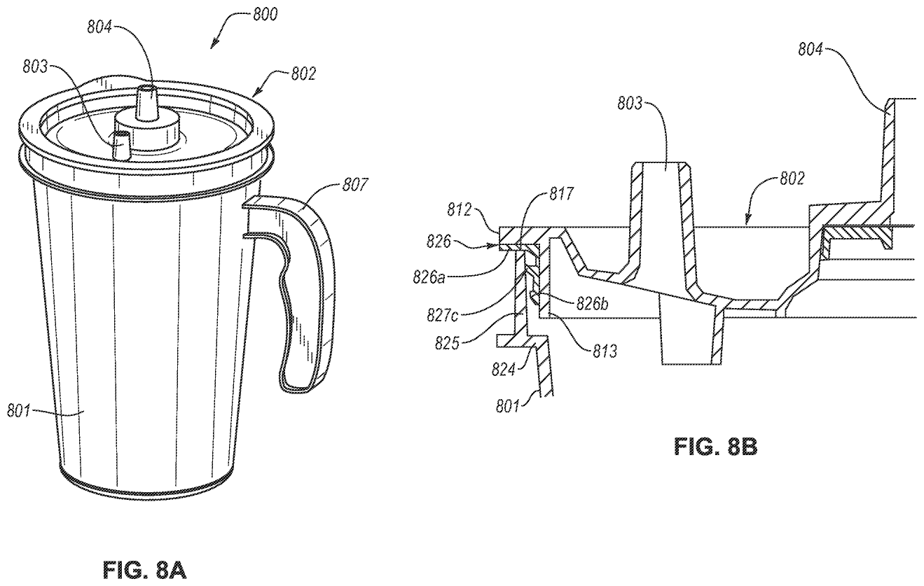

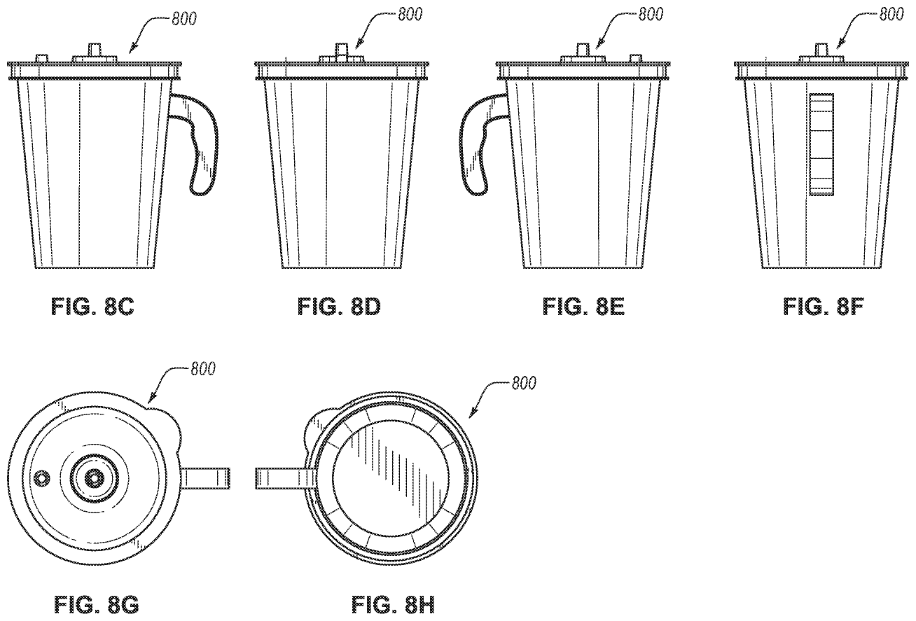

Turning to FIGS. 8A-8H, in some embodiments, a urine collection container assembly 800 may include a gasket 826 overmolded to one of a lid 801 or a canister 801 having an interior region. The lid 802 is secured or securable (e.g., mounted or mountable) to the canister 801 and including a vacuum port 804 configured to detachably secure to a first conduit to provide fluid communication between a pump and the interior region of the canister, and a urine port 803 configured to detachably secure to a second conduit to provide fluid communication between a urine collection device and the interior region of the canister. The canister 801 also may include at least one handle, such as a C- or L-shaped handle 807. In some embodiments, the urine collection container assembly 800 may include any of the handles, spouts, or other features of any of the urine collection container assemblies described herein.

In some embodiments, the canister 801 includes an upper region 825 having upper edge 817 and an inward surface. The lid 802 includes a flange 812 and a rim 813, according to an embodiment. The flange 812 may form an outer periphery of the lid 802 and include an inner surface. The rim 813 may be spaced inward from the outer periphery of the lid 802 and include an outer surface.

The gasket 826 may be overmolded (e.g., chemically bonded) to the lid 802 and positioned to interface a portion of the canister 801 to form a seal between the lid 802 and the canister 801 when the lid 802 is secured to the canister 801. For example, the gasket 826 may include a first portion 826a overmolded to the inner surface of the flange 812 and a second portion 826b overmolded to at least a portion of the outer surface of the rim 813 of the lid 802. The first portion 826a and the second portion 826b of the gasket 826 may be continuous with one another. The second portion 826b of the gasket 826 may include at least one fin 826c fin projecting outward from the second portion 826b of the gasket 826 and sized and positioned on the gasket 826 to interface with the inward surface of the upper region 825 of the canister 801. When the lid 802 is mounted or secured to the canister 801, at least some of the first portion 826a of the gasket 826 interfaces the upper edge 817 of the canister 801 and at least some (e.g., the fin 826c) of the second portion 826b of the gasket 826 interfaces the inward surface of the upper region 825 of the canister 801.

The gasket 826 may be chemically bonded to the lid 802. In some embodiments, the lid 802 includes a polypropylene material and the gasket 826 includes a thermoplastic elastomer material overmolded and chemically bonded to the polypropylene material of the lid 802.

Also contemplated herein is a method for forming a lid for a urine collection container assembly, such as the lid 802. The method may include a two-shot injection molding process such that the gasket and the lid are chemically inseparable following molding. The method may include injection molding, with a first material, a lid having a rim, a vacuum port configured to detachably secure to a first conduit to provide fluid communication between a pump and the interior region of the canister, and a urine port configured to detachably secure to a second conduit to provide fluid communication between a urine collection device and the interior region of the canister. The method also may include overmolding, with a second material, a gasket to at least a portion of the rim of the lid, the second material being different than the first material. Overmolding the gasket to at least a portion of the rim may include chemically bonding the gasket to the lid. In some embodiments, injection molding the lid may include injection molding the lid having the vacuum port, the urine port, a flange having an inner surface and forming an outer periphery of the lid, and the rim spaced inward from the outer periphery and having an outer surface. In some embodiments, overmolding the gasket to at least a portion of the rim includes overmolding a first portion of the gasket to the inner surface of the flange and overmolding a second portion of the gasket to at least a portion of the outer surface of the rim. In some embodiments, overmolding the second portion of the gasket to at least a portion of the outer surface of the rim includes overmolding the second portion of the gasket to at least a portion of the outer surface of the rim with a fin projecting outward from the second portion of the gasket. The first material of the lid may include a polypropylene material and the second material of the gasket may include a thermoplastic elastomer material. The acts of the method described above are for illustrative purposes. For example, the acts of the method can be performed in different orders, split into multiple acts, modified, supplemented, or combined. In an embodiment, one or more of the act of the method can be omitted from the method.

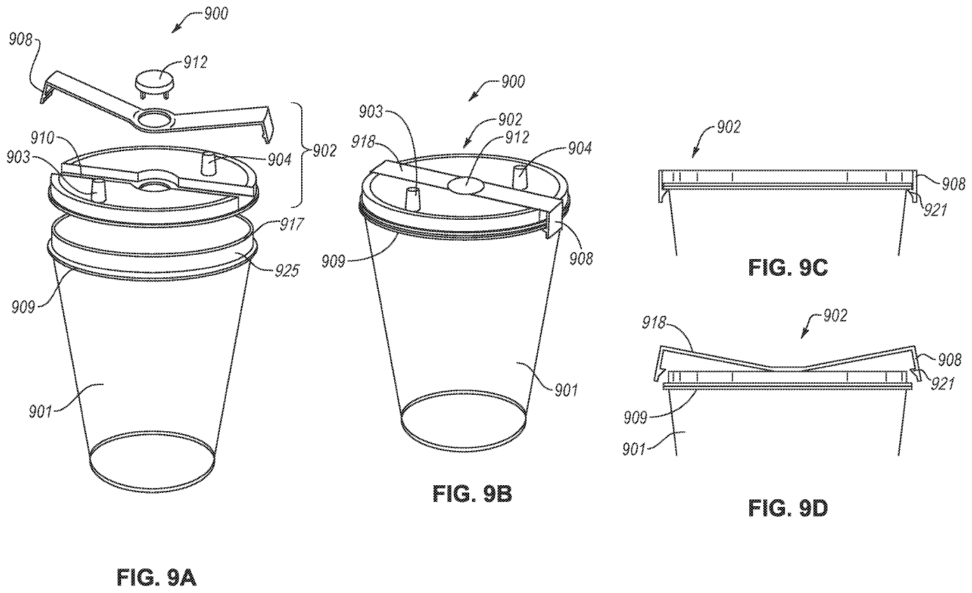

Turning now to FIGS. 9A-9D, in some embodiments of a urine collection container assembly, a lid may combine the advantages of a push button lid and clamps to secure and remove the lid. For example, a lid may include a clamp on one side or opposing sides of the lid that secure around the upper edge of the canister by pushing to seal the canister when clamped thereto. The lid also may include a button in the middle that connects to the clamps with lever arms. The lid may include a recessed cutout for the button and the lever arms such that when the lid is closed and locked, the top of the lid is substantially flat. The lever arms cooperatively connect or couple the button and the clamps such that when the button is pushed, both the clamps are released to unlock the lid. This configuration provides the technical effecting avoiding both spilling of urine and breaking of the canister, and also making usage of the assembly easier for individuals with lower dexterity or weakened muscles.

FIG. 9A illustrates an exploded view of a urine collection container assembly 900 including a canister 901 and a lid 902. The canister 901 may include an outer surface, an interior region, an upper region 925 having an upper edge 917, and a locking rib 909 on the outer surface of the canister 901 at least proximate to the upper region 925 of the canister 901.

In some embodiments, the lid 902 is secured or securable (e.g. mounted or mountable) to the canister 901 and includes a vacuum port 904 configured to detachably secure to a first conduit to provide fluid communication between a pump and the interior region of the canister 901, a urine port 903 configured to detachably secure to a second conduit to provide fluid communication between a urine collection device and the interior region of the canister 901, and at least one (e.g., two) clamp 908. The lid 902 also may include a button 912 and a recessed region 910. In some embodiments, the button 912 is disposed between (e.g., substantially central between) two clamps 908, and the recessed region 910 may extend from one side of the lid 902 to the other side of the lid 902.

The clamp 908 may include an inward oriented projection 921 configured to selectively engage with at least a portion of the locking rib 909 to secure the lid 902 to the canister 901. In some embodiments, the clamp 908 also includes at least one (e.g., two) lever arm 918 extending inward from the clamp 908. The lever arm 918 of each of the at least one clamp 908 is operably secured or securable to the button 912 clamps such that depressing the button 912 disengages the inward oriented projection 921 of each of the at least one clamp 908 from the locking rib 909 of the canister 901. The recessed region 910 of the lid may be complementary to the button 912 and the lever arm 918 of each of the two opposing clamps 908. When the inward oriented projection 921 of each of the two opposing clamps 908 is engaged with the locking rib 909 (shown in FIGS. 9B-9C), the button 912 and the lever arm 918 of each of the two opposing clamps 908 are generally planar with adjacent regions of the lid 902. When the inward oriented projection 921 of each of the two opposing clamps 908 is disengaged with the locking rib 909 (shown in FIG. 9D), the lever arm 918 of each of the two opposing clamps 908 may angle upward from the button 912 to the distal end of the clamp 908.

Also contemplated herein are urine collection systems comprising any of the urine collection container assemblies described herein and one or more (e.g., all) of a pump, a first conduit secured to the vacuum port on the lid and in fluid communication with the pump and the interior region of the canister, and/or a second conduit secured to the urine port on the lid and in fluid communication with the urine collection device and the interior region of the canister.

As used herein, the term “about” or “substantially” refers to an allowable variance of the term modified by “about” or “substantially” by ±10% or ±5%. Further, the terms “less than,” “or less,” “greater than,” “more than,” or “or more” include, as an endpoint, the value that is modified by the terms “less than,” “or less,” “greater than,” “more than,” or “or more.”

While various aspects and embodiments have been disclosed herein, other aspects and embodiments are contemplated. The various aspects and embodiments disclosed herein are for purposes of illustration and are not intended to be limiting.

1. A urine collection container assembly, comprising:
a canister having an interior region;
a lid secured or securable to the canister and including a vacuum port configured to detachably secure to a first conduit to provide fluid communication between a pump and the interior region of the canister, and a urine port configured to detachably secure to a second conduit to provide fluid communication between a urine collection device and the interior region of the canister; and
a gasket overmolded to the lid and positioned to interface a portion of the canister to form a seal when the lid is secured to the canister.

2. The urine collection container assembly of claim 1, wherein the gasket is chemically bonded to the lid.

3. The urine collection container assembly of claim 1, wherein:
the canister includes an upper edge and an inward surface;
the lid includes:
a flange having an inner surface and forming an outer periphery of the lid; and
a rim spaced inward from the outer periphery and having an outer surface;
the gasket includes a first portion overmolded to the inner surface of the flange and a second portion overmolded to at least a portion of the outer surface of the rim of the lid and continuous with the first portion of the gasket,
wherein when the lid is secured to the canister, at least some of the first portion of the gasket interfaces the upper edge of the canister and at least some of the second portion of the gasket interfaces the inward surface of the canister.

4. The urine collection container assembly of claim 3, wherein the second portion of the gasket includes a fin projecting outward from the second portion of the gasket and sized and positioned to interface with the inward surface of the canister.

5. The urine collection container assembly of claim 1, wherein the lid includes a polypropylene material and the gasket includes a thermoplastic elastomer material overmolded and chemically bonded to the polypropylene material of the lid.

6. The urine collection container assembly of claim 1, further comprising at least one handle on the canister.

7. The urine collection container assembly of claim 6, wherein the at least one handle includes at least one L- or C-shaped handle extending from an upper region of the canister.

8. The urine collection container assembly of claim 6, wherein the at least one handle includes opposing handles hingedly secured to the canister.

9. The urine collection container assembly of claim 6, wherein the at least one handle includes two opposing handles extending from an upper region of the canister.

10. The urine collection container assembly of claim 6, wherein the at least one handle includes a handle having two opposing end regions hingedly secured to opposing portions of an upper region of the canister.

11. The urine collection container assembly of claim 6, wherein the canister includes a spout and at least one of the rim of the lid and the second portion of the gasket define a gap or opening configured to selectively align with the spout to pour urine from the canister.

12. A urine collection system, comprising:
a urine collection container assembly including a canister having an interior region, a lid secured to the canister and including a vacuum port and a urine port, and a gasket overmolded to the lid and positioned to interface a portion of the canister to form a seal when the lid is secured to the canister;
a first conduit secured to the vacuum port;
a pump secured to the first conduit such that the first conduit provides fluid communication the pump and the interior region of the canister through the vacuum port;
a second conduit secured to the urine port;
a urine collection device secured to the second conduit such that the second conduit provides fluid communication between the urine collection device and the interior region of the canister through the urine port.

13. A method of forming a lid for a urine collection container assembly, the method comprising:
injection molding, with a first material a lid having a rim, a vacuum port configured to detachably secure to a first conduit to provide fluid communication between a pump and the interior region of the canister, and a urine port configured to detachably secure to a second conduit to provide fluid communication between a urine collection device and the interior region of the canister; and
overmolding, with a second material, a gasket to at least a portion of the rim of the lid, the second material being different than the first material.

14. The method of claim 13, wherein overmolding the gasket to at least a portion of the rim includes chemically bonding the gasket to the lid.

15. The method of claim 13, wherein:
injection molding the lid includes injection molding the lid having the vacuum port, the urine port, a flange having an inner surface and forming an outer periphery of the lid, and the rim spaced inward from the outer periphery and having an outer surface;
overmolding the gasket to at least a portion of the rim includes overmolding a first portion of the gasket to the inner surface of the flange and overmolding a second portion of the gasket to at least a portion of the outer surface of the rim.

16. The method of claim 15, wherein overmolding the second portion of the gasket to at least a portion of the outer surface of the rim includes overmolding the second portion of the gasket to at least a portion of the outer surface of the rim with a fin projecting outward from the second portion of the gasket.

17. The method of claim 13, wherein the first material of the lid includes a polypropylene material and the second material of the gasket includes a thermoplastic elastomer material.

18. A urine collection container assembly, comprising:
a canister having an outer surface, an interior region, an upper region having an upper edge, and a locking rib on the outer surface of the canister at least proximate to the upper region of the canister; and
a lid secured or securable to the canister and including a vacuum port configured to detachably secure to a first conduit to provide fluid communication between a pump and the interior region of the canister, a urine port configured to detachably secure to a second conduit to provide fluid communication between a urine collection device and the interior region of the canister, and at least one clamp having an inward oriented projection configured to selectively engage with at least a portion of the locking rib to secure the lid to the canister.

19. The urine collection container assembly of claim 18, wherein the at least one clamp includes two opposing clamps each having the inward oriented projection and a lever arm extending inward from the clamp, the lid further comprising a button operably secured or securable to the lever arm of each of the two opposing clamps between the two opposing clamps such that depressing the button disengages the inward oriented projection of each of the two opposing clamps from the locking rib of the canister.

20. The urine collection container assembly of claim 19, wherein the lid includes a recessed region complementary to the button and the lever arm of each of the two opposing clamps and when the inward oriented projection of each of the two opposing clamps is engaged with the locking rib, the button and the lever arm of each of the two opposing clamps are generally planar with adjacent regions of the lid.

21. The urine collection container assembly of claim 18, wherein:
the at least one clamp includes two opposing clamps hingedly secured to the lid;
the lid includes:
a flange forming an outer periphery of the lid that extends outward beyond the upper edge of the canister when the lid is secured to the canister;
a rim spaced inward from the outer periphery of the flange;
the canister includes a shelf spaced from the upper edge of the canister and projecting inward;
the urine collection container assembly further includes a seal disposed on the shelf of the canister, wherein the seal is disposed between the rim and the shelf of the canister when the lid is secured to the canister with the rim disposed at least partially within the canister.

22. The urine collection container assembly of claim 18, wherein:
the at least one clamp includes two opposing clamps hingedly secured to the lid;
the lid includes a gasket secured thereto and disposed between the upper edge of the canister and the lid when the canister is secured to the lid.

23-28. (canceled)
